SEQUENCE LISTING (13 sequences)

SEQ ID NO: 1 - protein, 20 aa, human
AYIFIDEAHITQALIWLSQR

SEQ ID NO: 2 - protein, 18 aa, human
LLIYAVLPTGDVIGDSAK

SEQ ID NO: 3 - protein, 18 aa, human
LLLQQVSLPELPGEYSMK

SEQ ID NO: 4 - protein, 11 aa, human
QGLLPVLESFK

SEQ ID NO: 5 - protein, 14 aa, human
LLDNWDSVTSTFSK

SEQ ID NO: 6 - protein, 17 aa, human
KEPSQGTTTFAVTSILR

SEQ ID NO: 7 - protein, 15 aa, human
VFAIPPSFASIFLTK

SEQ ID NO: 8 - protein, 17 aa, human
QEPSQGTTTFAVTSILR

SEQ ID NO: 9 - protein, 10 aa, human
WLQGSQELPR

SEQ ID NO: 10 - protein, 26 aa, human
LWAYLTIQQLLEQTVSASDADQQALR

SEQ ID NO: 11 - protein, 13 aa, human
AEAQAQYSAAVAK

SEQ ID NO: 12 - protein, 30 aa, human
YSQPEDSLIPFFEITVPESQLTVSQFTLPK

SEQ ID NO: 13 - protein, 13 aa, human
IAIANIIDEIIEK

(19) United States
(12) Patent Application Publication
(10) Pub. No.: US 2007/0105149 A1
(43) Pub. Date: May 10, 2007

(54) Assay for diabetes

(75) Inventors: Bradley John Walsh, South Curl Curl (AU); Geoffrey Bruce Randall, Glenorie (AU); Steven Brian Wilson, North Turrmurra (AU)

Correspondence Address: BARNES & THORNBURG LLP, P.O. BOX 2786, CHICAGO, IL, 60690-2786, US

(73) Assignee: MINOMIC PTY LTD, New South Wales (AU)

(21) Appl. No.: 10/570,836

(22) PCT Filed: Sep. 6, 2004
(86) PCT No.: PCT/AU04/01202

§ 371 Date: Dec. 13, 2006

(30) Foreign Application Priority Data
Sep. 5, 2003 (AU) 2003904870

(51) Int. Cl. G01N 33/53; C07K 16/18

(52) U.S. Cl. 435/7.1; 530/388.25

(57) ABSTRACT

An assay for testing a subject for diabetes or a predisposition to diabetes including analysing a biological fluid from a subject for the presence of one or more proteins selected from the group consisting of Alpha 2 macroglobulin, Apolipoprotein All, Immunoglobulin alpha heavy chain constant region, Immunoglobulin mu chain C region, Chain A of Human IgA1, Inter-alpha-trypsin inhibitor heavy chain H4 precursor, and Apolipoprotein B100; wherein detection of the protein is indicative of diabetes or a predisposition to diabetes in the subject

### TECHNICAL FIELD

The present invention relates to assays for detecting the presence of indicator proteins or peptides in biological samples to screen for diabetes or identify a predisposition to diabetes in a subject.

### BACKGROUND ART

Diabetes mellitus is a syndrome which results in disregulation of glucose homeostasis with multiple etiologic factors that generally involve absolute or relative insulin deficiency or insulin resistance or both. All causes of diabetes ultimately lead to hyperglycemia, which is the hallmark of this disease syndrome. Several clinical subclasses are recognized, including: Type I (insulin-dependent or IDDM), Type II (non-insulin-dependent diabetes mellitus), maturity-onset diabetes of the young (MODY) and gestational diabetes.

Overall, in the United States the prevalence of diabetes is about 2 to 4 percent, with IDDM comprising 7 to 10 percent of all cases. The prevalence of IDDM is probably more accurate than the estimates for Type II diabetes. This is due at least in part to the relative ease of ascertainment of IDDM, while many patients with Type II diabetes are asymptomatic and thus this form of the disease goes undiagnosed. Type II diabetes, the most common form of diabetes found in the United States, is characterized by a later age of onset, insulin resistance and impaired insulin secretion. Obesity and increased hepatic glucose output are also associated with Type II diabetes. Indeed, in the United States, 80 to 90 percent of Type II diabetes patients are obese. The precise role of obesity in the causes of Type II diabetes and the development of complications associated with diabetes remains equivocal.

Type II diabetes has been shown to have a strong familial transmission: 40% of monozygotic twin pairs with Type II diabetes also have one or several first degree relatives affected with the disease. Barnett et al. (1981) Diabetologia 20:87-93. In the Pima Indians, the relative risk of becoming diabetic is increased twofold for a child born to one parent who is diabetic, and sixfold when both parents are affected (Knowler, W. C., et al. (1988) Genetic Susceptibility to Environmental Factors. A Challenge for Public Intervention, Almquist & Wiksele International: Stockholm. p. 67-74). Concordance of monozygotic twins for Type II diabetes has been observed to be over 90%, compared with approximately 50% for monozygotic twins affected with Type I diabetes (Barnett, A. H., et al. (1981) Diabetologia 20(2):87-93). Non-diabetic twins of Type II diabetes patients were shown to have decreased insulin secretion and a decreased glucose tolerance after an oral glucose tolerance test (Barnett, A. H., et al. (1981) Brit. Med. J. 282:1656-1658).

Central fat, particularly intra-abdominal adipose tissue (IAAT), is associated with increased risk for Type II diabetes (Vague, J. (1996) Obesity Res. 4(2):201-3; Kissebah, A. H., et al. (1982) J. of Clinical Endocrinology & Metabolism 54(2):254-60; Bjomtorp, P. (1992) Obesity 579-586).

Diabetes is a complex syndrome affected not only by familial transmission but by environmental factors as well (Kahn, C. R. et al. (1996) Ann. Rev. of Med. 47:509-31; Aitman, T. J. and Todd, A. J. (1995) Baillieres Clin. Endocrinology & Metabolism 9(3):631-56). There is a high prevalence of the disease in world populations. Expression is strongly age-dependent and the etiology is heterogeneous. The high prevalence of the disease in world populations, reduced penetrance, and the presence of phenocopies each contributes to reducing the power of linkage studies. Sib pair studies and the transmission disequilibrium test, non-parametric methods which do not require a model for mode of inheritance, are hampered by heterogeneity and the large number of phenocopies expected for such a complex common disease. A number of published findings suggest linkage of diabetes to chromosome 20q (Ji et al. (1997) Diabetes 46:876-81; Bowden, D. W., et al. (1997) Diabetes 46:882-86; Velho et al. (1997) Diabetes and Metabolism 23:34-37; and Zouali et al. (1997) Human Molec. Genet. 6:1401-1408), but definition of a locus linked to susceptibility to Type II diabetes has thus far been unsuccessful.

Every year throughout the world thousands of people die and many thousands more suffer heart and kidney problems, stroke or lose a limb or their vision as a result of Type II diabetes.

Type II diabetes diagnosis and management, for example, is currently hampered by a number of deficiencies. Three areas where better testing is desirable are initial diagnosis, monitoring of blood glucose control, and better monitoring of renal damage.

Urine should potentially be a rich source of biomarkers. For proteomics research, however, the presence of high amounts of salts such as urea have made study difficult. There are a number of other tests being used to diagnose Type II diabetes but none of these is ideal. There are deficiencies in each test that are multifactorial. In many cases, patients do not want to give blood or return for further testing and produce multiple samples such as blood and urine.

In Australia, 15% of 55-65 year olds have Type II diabetes but approximately 50% are undiagnosed due to the reluctance of doctors to order a glucose tolerance test. This test requires a blood sample then a dose of glucose orally followed by taking another blood sample 2 hours later. A simpler, less invasive test would be commercially very attractive. Furthermore, children who have symptoms of diabetes are usually diagnosed with Type I diabetes. This is of particular concern given the rise in childhood Type II diabetes, and some centers report a misdiagnosis in up to 25% of cases.

Currently, blood glucose control is monitored by the glycosylated haemoglobin test. This the test is complicated by anything that changes the half-life of red cell turnover. A test that shows efficacy in monitoring blood glucose control in the 2-3 day or 1 week period would be highly desirable.

All diabetics should be monitored once a year for renal damage via urine collection. This is not done for around 70% of patients due to compliance issues.

As the number of people with diabetes grows worldwide, the disease takes an ever-increasing proportion of national health care budgets. Without primary prevention, the diabetes epidemic will continue to grow. Even worse, diabetes is projected to become one of the world's main disablers and killers within the next twenty-five years. Immediate action is needed to reduce the onset of diabetes and to introduce more cost-effective diagnostic strategies to reverse this trend.

The present inventors have now identified new protein and peptide markers which are useful in developing non-invasive assays for diabetes.

### DISCLOSURE OF INVENTION

In a first aspect, the present invention provides an assay for testing a subject for diabetes or a predisposition to diabetes comprising:

analysing a biological fluid from a subject for the presence of one or more proteins selected from the group consisting of Alpha 2 macroglobulin, Apolipoprotein A1, Immunoglobulin alpha heavy chain constant region, Immunoglobulin mu chain C region, Chain A of Human IgA1, Inter-alpha-trypsin inhibitor heavy chain H4 precursor, and Apolipoprotein B-100;

wherein detection of the protein is indicative of diabetes or a predisposition to diabetes in the subject.

Preferably, Alpha 2 macroglobulin is detected by the presence of a peptide selected from one or more of the following:

| | |
|---|---|
| AYIFIDEAHITQALIWLSQR | (SEQ ID NO:1) |
| LLIYAVLPTGDVIGDSAK | (SEQ ID NO:2) |
| LLLQQVSLPELPGEYSMK | (SEQ ID NO:3) |

Preferably, Apolipoprotein A1 is detected by the presence of a peptide selected from one or more of the following:

| | |
|---|---|
| QGLLPVLESFK | (SEQ ID NO:4) |
| LLDNWDSVTSTFSK | (SEQ ID NO:5) |

Preferably, Immunoglobulin alpha heavy chain constant region is detected by the presence of the following peptide:

| | |
|---|---|
| KEPSQGTTTFAVTSILR | (SEQ ID NO:6) |

Preferably, Immunoglobulin mu chain C region is detected by the presence of the following peptide:

| | |
|---|---|
| VFAIPPSFASIFLTK | (SEQ ID NO:7) |

Preferably, Chain A of Human IgA1 is detected by the presence of a peptide selected from one or more of the following:

| | |
|---|---|
| QEPSQGTTTFAVTSILR | (SEQ ID NO:8) |
| WLQGSQELPR | (SEQ ID NO:9) |

Preferably, Inter-alpha-trypsin inhibitor heavy chain H4 precursor is detected by the presence of a peptide selected from one or more of the following:

| | |
|---|---|
| LWAYLTIQQLLEQTVSASDADQQALR | (SEQ ID NO:10) |
| AEAQAQYSAAVAK | (SEQ ID NO:11) |

Preferably, Apolipoprotein B-100 is detected by the presence of a peptide selected from one or more of the following:

| | |
|---|---|
| YSQPEDSLIPFFEITVPESQLTVSQFTLPK | (SEQ ID NO:12) |
| IAIANIIDEIIEK | (SEQ ID NO:13) |

It will be appreciated that the proteins found by the present inventors as being indicative of diabetes or a predisposition to diabetes may be identified by detecting the whole protein or fragments thereof in a biological fluid.

The biological fluid can be any suitable fluid such as urine, saliva, blood, blood products such as serum, plasma, tears, cerebrospinal fluid, and lymph. The biological fluid can be assayed neat or concentrated or fractionated prior to assaying.

Preferably, the biological fluid is urine.

In one preferred form, proteins present in the biological sample are digested to form peptide fragments which are detected by conducting mass spectrophotometric analysis on the sample in a manner effective to maximize elucidation of discernible peptide fragments contained therein; and comparing mass spectrum profiles of peptides consisting of amino acid residues of SEQ ID NOS:1 to 13 to mass spectrum profiles of peptides elucidated from said sample; wherein recognition of a mass spectrum profile in the sample displaying the mass spectrum profile for any one or more of peptides having amino acid residues of SEQ ID NOS:1 to 13 is indicative of diabetes or a predisposition to diabetes.

In another preferred form, an antibody which recognises a peptide having amino acid residues of one of SEQ ID NOS: 1 to 13 is used to probe the sample for the presence of one or more of the proteins Alpha 2 macroglobulin, Apolipoprotein A1, Immunoglobulin alpha heavy chain constant region, Immunoglobulin mu chain C region, Chain A of Human IgA1, Inter-alpha-trypsin inhibitor heavy chain-H4 precursor, and Apolipoprotein B-100.

The present inventors have found that urine from Type II diabetics have detectable levels of one or more proteins selected from Alpha 2 macroglobulin, Apolipoprotein A1, Immunoglobulin alpha heavy chain constant region, Immunoglobulin mu chain C region, Chain A of Human IgA1, Inter-alpha-trypsin inhibitor heavy chain H4 precursor, and Apolipoprotein B-100.

Preferably, the subject is a human.

In a second aspect, the present invention provides an isolated protein, protein fragment or peptide detectable in a biological sample of a subject being indicative of diabetes or a predisposition to diabetes in a subject, the protein, protein fragment or peptide comprises or contains a peptide marker having one or more of the following amino acid sequences:

| | |
|---|---|
| AYIFIDEAHITQALIWLSQR, | (SEQ ID NO:1) |
| LLIYAVLPTGDVIGDSAK, | (SEQ ID NO:2) |
| LLLQQVSLPELPGEYSMK, | (SEQ ID NO:3) |
| QGLLPVLESFK, | (SEQ ID NO:4) |
| LLDNWDSVTSTFSK, | (SEQ ID NO:5) |
| KEPSQGTTTFAVTSILR, | (SEQ ID NO:6) |
| VFAIPPSFASIFLTK, | (SEQ ID NO:7) |
| QEPSQGTTTFAVTSILR, | (SEQ ID NO:8) |
| WLQGSQELPR, | (SEQ ID NO:9) |
| LWAYLTIQQLLEQTVSASDADQQALR, | (SEQ ID NO:10) |
| AEAQAQYSAAVAK, | (SEQ ID NO:11) |
| YSQPEDSLIPFFEITVPESQLTVSQFTLPK, | (SEQ ID NO:12) |
| or | |
| IAIANIIDEIIEK. | (SEQ ID NO:13) |

Preferably, the peptide marker is selected from the group consisting of:

| | |
|---|---|
| AYIFIDEAHITQALIWLSQR, | (SEQ ID NO:1) |
| LLIYAVLPTGDVIGDSAK, | (SEQ ID NO:2) |
| LLLQQVSLPELPGEYSMK, | (SEQ ID NO:3) |
| QGLLPVLESFK, | (SEQ ID NO:4) |
| LLDNWDSVTSTFSK, | (SEQ ID NO:5) |
| KEPSQGTTTFAVTSILR, | (SEQ ID NO:6) |
| VFAIPPSFASIFLTK, | (SEQ ID NO:7) |
| QEPSQGTTTFAVTSILR, | (SEQ ID NO:8) |
| WLQGSQELPR, | (SEQ ID NO:9) |
| LWAYLTIQQLLEQTVSASDADQQALR, | (SEQ ID NO:10) |
| AEAQAQYSAAVAK, | (SEQ ID NO:11) |
| YSQPEDSLIPFFEITVPESQLTVSQFTLPK, | (SEQ ID NO:12) |
| and | |
| IAIANIIDEIIEK. | (SEQ ID NO:13) |

The peptides according to the invention were obtained by:
(a) concentrating/fractionating urine samples from diabetics and healthy individuals;
(b) separating proteins present in the concentrated urine samples; and
(c) identifying protein, protein fragment or peptide present in the urine of diabetics but absent or undetectable in healthy individuals.

The urine is preferably concentrated/fractionated by membrane-based electrophoresis. Alternatively urine can be concentrated by precipitation using acetone and/or trichloroacetic acid. It will be appreciated that other forms of concentration known to the art could also be used in this regard.

The peptides can be separated by chromatography and identified by mass spectrometry.

In a third aspect, the present invention provides an isolated antibody directed to a protein, protein fragment or peptide detectable in a biological sample of a subject being indicative of diabetes or a predisposition to diabetes in a subject according to the second aspect of the present invention.

In one preferred from, the antibody is a polyclonal antibody which is derived by immunising mice or other suitable animal with one or more proteins, protein fragments or peptides according to the first aspect of the present invention.

In another preferred form, for the antibody is an isolated monoclonal antibody to one or more proteins, protein fragments or peptides according to the first aspect of the present invention. Methods for developing monoclonal antibodies are well known to the art.

It will be appreciated that when an animal has raised an immune response to one or more peptides according to the first aspect of the present invention, hyperimmune serum or ascites fluid, for example, can be collected by usual methods. Specific antibodies can be obtained by separation methods known to the art such as precipitation, affinity chromatography, Protein A separation. The separated sera or ascites fluid can be used whole, diluted or as a starting material for separation of one or more peptides according to the first aspect of the present invention.

In one preferred from, the antibodies are detectably labelled. In one preferred form, the label is fluorochrome fluoresein isothiocyanate (FITC). Other labels such as Texas Red, Oregon Green, TRITC, Alexa dyes, allophycocyanin or rhodamine would also be suitable for the present invention. In another preferred form, the antibodies are radioactively labelled.

The assay may be an ELISA assay or radioassay. Other suitable assays utilizing antibodies are well known to the art and include protein chip based matrices.

In a fourth aspect, the present invention provides an assay for testing a subject for diabetes or a predisposition to diabetes comprising:

obtaining a urine sample from a subject;

concentrating the urine sample;

digesting proteins present in the concentrated urine sample to form peptides; optionally, separating the peptides; and

analysing the peptides for the presence of one or marker peptides having an amino acid sequence of SEQ ID NOS:1 to 13, wherein the presence of marker peptides having an amino acid sequence of SEQ ID NOS:1 to 13 is indicative of diabetes or a predisposition to diabetes in the subject.

Preferably, the one or more proteins, protein fragments or peptides are detected by the use of an antibody according to third aspect of the present invention.

In a fifth aspect, the present invention provides a kit for assaying a subject for diabetes or a predisposition to diabetes comprising:
(a) one or more antibodies according to the third aspect of the present invention; and
(b) suitable reagents and diluents for the assay.

In a sixth aspect, the present invention provides use of a marker peptide having an amino acid sequence of any one of SEQ ID NOS:1 to 13 in an assay for diabetes or a predisposition to diabetes.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia prior to development of the present invention.

In order that the present invention may be more clearly understood, preferred forms will be described with reference to the following examples.

### MODE(S) FOR CARRYING OUT THE INVENTION

Materials and Methods

Before describing the preferred embodiments in detail, the principal of operation of a membrane-based electrophoresis apparatus will first be described. An electric field or potential applied to ions in solution will cause the ions to move toward one of the electrodes. If the ion has a positive charge, it will move toward the negative electrode (cathode). Conversely, a negatively-charged ion will move toward the positive electrode (anode).

In the apparatus used for present invention, ion-permeable barriers that substantially prevent convective mixing between the adjacent chambers of the apparatus or unit are placed in an electric field and a cell type or population in the sample is selectively transported through an ion-permeable barrier. The particular ion-permeable barriers used will vary for different applications and generally have characteristic average pore sizes and pore size distributions and/or isoelectric points allowing or substantially preventing passage of different components.

Gradiflow™ Apparatus

A number of membrane-based electrophoresis apparatus have been developed by, or in association with, Gradipore Limited, Australia. The apparatus are marketed and used under the name Gradiflow™. In summary, the apparatus typically includes a cartridge which houses a number of membranes forming at least two chambers, cathode and anode in respective electrode chambers connected to a suitable power supply, reservoirs for samples, buffers and electrolytes, pumps for passing samples, buffers and electrolytes, and cooling means to maintain samples, buffers and electrolytes at a required temperature during electrophoresis. The cartridge contains at least three substantially planar membranes disposed and spaced relative to each other to form two chambers through which sample or solvent can be passed. A separation membrane is disposed between two outer membranes (termed restriction membranes as their molecular mass cut-offs are usually smaller than the cut-off of the separation membrane). When the cartridge was installed in the apparatus, the restriction membranes are located adjacent to an electrode. The cartridge is described in AU 738361. Description of membrane-based electrophoresis can be found in U.S. Pat. No. 5,039,386 and U.S. Pat. No. 5,650,055 in the name of Gradipore Limited, incorporated herein by reference. An apparatus particularly suitable for use in isoelectric separation applications can be found in WO 02/24314 in the name of The Texas A&M University System and Gradipore Limited, incorporated herein by reference.

The electrophoresis apparatus used in urine separation comprised:
(a) a first electrolyte chamber
(b) a second electrolyte chamber,
(c) a first sample chamber disposed between the first electrolyte chamber and the second electrolyte chamber
(d) a second sample chamber disposed adjacent to the first sample chamber disposed and between the first electrolyte chamber and the second electrolyte chamber;
(e) a first ion-permeable barrier disposed between the first sample chamber and the second sample chamber, the first ion-permeable barrier prevents substantial convective mixing of contents of the first and second sample chambers;
(f) a second ion-permeable barrier disposed between the first electrolyte chamber and the first sample chamber, the second ion-permeable barrier prevents substantial convective mixing of contents of the first electrolyte chamber and the first sample chamber;
(g) a third ion-permeable barrier disposed between the second sample chamber and the second electrolyte chamber, the third ion-permeable barrier prevents substantial convective mixing of contents of the second electrolyte chamber and the second sample chamber; and
(h) electrodes disposed in the first and second electrolyte chambers.

The electrophoresis apparatus may further comprise one or more of:
(i) an electrolyte reservoir;
(j) a first sample reservoir and a second sample reservoir;
(k) means for supplying electrolyte from the electrolyte reservoir to the first and second electrolyte chambers; and
(l) means for supplying sample or liquid from at least the first sample reservoir to the first sample chamber, or from the second sample reservoir to the second sample chamber.

The apparatus may comprise:
(m) a first electrolyte reservoir and a second electrolyte reservoir; and
(n) means for supplying electrolyte from the first electrolyte reservoir to the first electrolyte chamber and electrolyte from second electrolyte reservoir to the second electrolyte chamber.

The apparatus may further comprise one or more of:

means for circulating electrolyte from the electrolyte reservoir(s) through the electrolyte chambers forming electrolyte streams in the electrolyte chambers; and

means for circulating contents from each of the first and second sample reservoirs through the respective first and second sample chambers forming first and second sample streams in the respective sample chambers;

means for removing and replacing sample in the first or second sample reservoirs; and

means to maintain temperature of electrolyte and sample solutions.

All ion-permeable barriers were membranes having a characteristic average pore size and pore size distribution.

The electrophoresis apparatus contained a separation unit housing the chambers and ion-permeable barriers which is provided as a cartridge or cassette fluidly connected to the electrolyte reservoir(s) and the sample reservoirs.

In use, the urine sample to be separated was placed in the first or second sample chamber. Electrolyte was placed in the first and second electrolyte chambers. Electrolyte or other liquid can be placed in the first and/or second sample chamber. An electric potential was applied to the electrodes and some urine proteins in the first and/or second sample chamber were caused to move through a diffusion barrier to the second and/or first sample chamber.

Urine Samples

Fifty millilitres of morning urine are collected from Type II diabetic patients and age matched controls. Protein membrane separations were performed with a Gradiflow BF400 apparatus and the protein separation product concentrated 10 times using a standard Acetone-HCl precipitation before freezing at −80° C.

Gradiflow™ Separation

Two protein separations are performed using the Gradiflow BF400 and Tris/EACA/EDTA buffer solution (46.3 g Tris and 5.24 g EACA and 1 mM EDTA in 2 l MilliQ water). The first separation at 250V for 4 hours with a 5-125-5 cartridge (restriction membrane—separation membrane—restriction membrane cut off). The separation product was then used for a second separation, this time with a 5-25-5 kDa cut-off cartridge (250 V, 4 hr). The final product was then concentrated before being stored at −80° C.

Acetone or TCA Precipitation

Precipitate sample in either 20% trichloroacetic acid (TCA) (20 g of TCA made up to 100 ml with MilliQ water) or acetone. In each case, add 1 part urine to 4 parts TCA or acetone. Store for 2 hours at −20° C. If using TCA, then centrifuge at 25,000 g for 15 minutes. Resuspend supernatant in 1 ml cold acetone. Then for TCA or acetone methods centrifuge 25000 g for 15 min. Vacuum dry pellet for 5 to 15 minutes, until no liquid remains. Resuspend pellet in the buffer required for the following process.

Trypsin Digestion

Protein was dialysed overnight against water with a 1 kDa cut-off membrane and proteins evaporated to dryness. Samples were resuspended in 1 M Urea, 50 mM NH₄HCO₃ and 5 mM CaCl₂. Trypsin was added at an enzyme to protein ration of 1:50 and the reaction incubated at 37° C. for 15 h. The peptide digests were evaporated to dryness and resuspended in water to a concentration of 1 μg/μl.

Mass Spectrometry

The peptide mixture was filtered and 1 μg loaded onto a micro C18 precolumn After a 10 min wash the pre-column is switched in line with an analytical column containing C18 RP silica. Peptides were eluted using a linear gradient of H₂O:CH₃CN (95:5, 0.1% formic acid-buffer A) to H₂O:CH₃CN (40:60, 0.1% formic acid-buffer B) at 200 nl/min over 30 min. The column was connected via a fused silica capillary to a low volume tee (Upchurch Scientific) where high voltage (2300 V) is applied and a nano electrospray needle is positioned ˜1 cm from the orifice of a tandem mass spectrometer (either Waters Q-TOF or Applied biosystems Q-Star). Positive ions were generated by electrospray and the mass spectrometer operated in information dependent acquisition mode (IDA). Tandem mass spectra are accumulated for 2 s (m/z 50-2000) and processing scripts are sued to automatically determine peptide sequence.

Antibodies

A monoclonal antibody specific against a target peptide or protein according to the present invention may be produced, for example, by the polyethylene glycol (PEG) mediated cell fusion method, in a manner well-known in the art.

Traditionally, monoclonal antibodies have been made according to fundamental principles laid down by Kohler and Milstein. Mice are immunized with antigens, with or without, adjuvants. The splenocytes are harvested from the spleen for fusion with immortalized hybridoma partners. These are seeded into microtitre plates where they can secrete antibodies into the supernatant that is used for cell culture. To select from the hybridomas that have been plated for the ones that produce antibodies of interest the hybridoma supernatants are usually tested for antibody binding to antigens in an ELISA (enzyme linked immunosorbent assay) assay. The wells that contain the hybridoma of interest will contain antibodies that will bind most avidly to the test antigen, usually the immunizing antigen. The cells in these wells are then subcloned in limiting dilution fashion to produce monoclonal hybridomas. The selection for the clones of interest is repeated using an ELISA assay to test for antibody binding. Therefore, the principle that has been propagated is that in the production of monoclonal antibodies the hybridomas that produce the most avidly binding antibodies are the ones that are selected from among all the hybridomas that were initially produced. The preferred antibody is the one with highest affinity for the antigen of interest.

There have been many modifications of this procedure such as using whole cells for immunization. In this method, instead of using purified antigens, entire cells are used for immunization. Another modification is the use of cellular ELISA for screening. In this method instead of using purified antigens as the target in the ELISA, fixed cells are used. In addition to ELISA tests, complement mediated cytotoxicity assays have also been used in the screening process. However, antibody-binding assays were used in conjunction with cytotoxicity tests. Thus, despite many modifications, the process of producing monoclonal antibodies relies on antibody binding to the test antigen as an endpoint.

The purified monoclonal antibody is utilized for immunochemical studies and for diagnostic assays and the like.

Polyclonal antibody production and purification utilizing one or more animal hosts in a manner well-known in the art can be performed by a skilled artisan.

Assays

The peptide markers of the present invention may be used as antigens in immunoassays for the detection of those individuals suffering from the disease known to be evidenced by said marker sequence. Such assays may include but are not limited to: radioimmunoassay, enzyme-linked immunosorbent assay (ELISA), “sandwich” assays, precipitin reactions, gel diffusion immunodiffusion assay, agglutination assay, fluorescent immunoassays, protein A or G immunoassays and immunoelectrophoresis assays.

Monoclonal or polyclonal antibodies produced against the peptide markers are useful in an immunoassay on samples of blood or blood products such as serum, plasma or the like, spinal fluid or other body fluid, e.g. saliva, urine, lymph, and the like, to diagnose patients with the characteristic disease state linked to ther marker sequence. The antibodies can be used in any type of immunoassay. This includes both the two-site sandwich assay and the single site immunoassay of the non-competitive type, as well as in traditional competitive binding assays.

For ease and simplicity of detection, and its quantitative nature, the sandwich or double antibody assay of which a number of variations exist, all of which are contemplated by the present invention. For example, in a typical sandwich assay, unlabelled antibody is immobilized on a solid phase such as microtiter plate, and the sample to be tested is added. After a certain period of incubation to allow formation of an antibody-antigen complex, a second antibody, labelled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which be quantitated by comparison with control samples containing known amounts of antigen.

### RESULTS

Peptides sequences were compared between diabetic and non-diabetic patients. Table 1 provides a list of peptides occurring in diabetic samples only.

Forty samples were tested comprising urine collected from twenty non-diabetic and twenty diabetic donors. The differences between the normals and diabetics were seen in the proteins Alpha 2 macroglobulin, Apolipoprotein A1, Immunoglobulin alpha heavy chain constant region, Immunoglobulin mu chain C region, Chain A of Human IgA1, Inter-alpha-trypsin inhibitor heavy chain H4 precursor, and Apolipoprotein B-100.

TABLE 1

| | | Database |
|---|---|---|
| | | Accession |
| Peptide | Protein | No |
| AYIFIDEAHITQALIWLSQR | Alpha 2 macroglobulin | CAA01533 |
| (SEQ ID NO: 1) | | |
| LLIYAVLPTGDVIGDSAK | Alpha 2 macroglobulin | CAA01533 |
| (SEQ ID NO: 2) | | |
| LLLQQVSLPELPGEYSMK | Alpha 2 macroglobulin | CAA01533 |
| (SEQ ID NO: 3) | | |
| QGLLPVLESFK | Apolipoprotein A1 | CAA00975, |
| (SEQ ID NO: 4) | | 1AV1A |
| LLDNWDSVTSTFSK | Apolipoprotein A1 | CAA00975, |
| (SEQ ID NO: 5) | | 1AV1A |
| KEPSQGTTTFAVTSILR | Immunoglobulin alpha | AAK72411 |
| (SEQ ID NO: 6) | heavy chain constant | |
| | region | |
| VFAIPPSFASIFLTK | Immunoglobulin mu | MHHU, |
| (SEQ ID NO: 7) | chain C region | MHHUBT |
| QEPSQGTTTFAVTSILR | Chain A of Human Iga1 | 1IGA_A |
| (SEQ ID NO: 8) | | |
| WLQGSQELPR | Chain A of Human Iga1 | 1IGA_A |
| (SEQ ID NO: 9) | | |
| LWAYLTIQQLLEQTVSASD | Inter-alpha-trypsin | ITH4_{—} |
| ADQQALR | inhibitor heavy chain | HUMAN, |
| (SEQ ID NO: 10) | H4 precursor | HCHU |
| AEAQAQYSAAVAK | Inter-alpha-trypsin | ITH4_{—} |
| (SEQ ID NO: 11) | inhibitor heavy chain | HUMAN, |
| | H4 precursor | HCHU |
| YSQPEDSLIPFFEITVPES | Apolipoprotein B-100 | LPHUB |
| QLTVSQFTLPK | | |
| (SEQ ID NO: 12) | | |
| IAIANIIDEIIEK | Apolipoprotein B-100 | LPHUB |
| (SEQ ID NO: 13) | | |

Products to be derived from this test are likely to be ELISA based immunoassays, or protein chips or nanotechnology based ion channel switching (ICS) capable of measuring the levels of the proteins/peptides singly or in a combination from urine or other biological fluids including plasma and tears. Another test would be to use a mass spectrometer to determine the presence and amounts of the peptides from a mixture of urine or other biological fluid.

Methods for Analysing the Proteins/Peptides

Using either unprocessed sample or following depletion of high abundance proteins using immunodepletion techniques (eg Seppro-Tm (GenWay Biotech, Calif., USA)),

Methods for analysing the proteins/peptides include:

two dimensional gel electrophoresis, followed by comparison of gel images, excision of protein spots and their subsequent digestion using enzyme such as trypsin. The resulting peptide digest can then analysed by mass spectrorhetry using either MALDI or tandem mass spectrometry.

Alternatively, the entire sample may be digested and analysed by liquid chromatography (single or two dimensional incorporating ion exchange and reverse phase chromatography) and mass spectrometry (tandem MS).

Alternatively samples could be analysed using protein chips of the like used in Surface Enhanced Laser Desorption Ionisation (SELDI™).

Detection Threshold

The threshold for positive detection of the peptides/proteins by many tests is approximately 50 femtomoles. The sensitivity of a given test will have an effect on the detection limit for any given peptide marker.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An assay for testing a subject for diabetes or a predisposition to diabetes comprising:
analysing a biological fluid from a subject for the presence of one or more proteins selected from the group consisting of Alpha 2 macroglobulin, Apolipoprotein A1, Immunoglobulin alpha heavy chain constant region, Immunoglobulin mu chain C region, Chain A of Human IgA1, Inter-alpha-trypsin inhibitor heavy chain H4 precursor, and Apolipoprotein B-100;
wherein detection of the protein is indicative of diabetes or a predisposition to diabetes in the subject.

2. The assay according to claim 1 wherein the one or more proteins are detected by the presence of a peptide marker selected from the group consisting of:

| | |
|---|---|
| AYIFIDEAHITQALIWLSQR, | (SEQ ID NO:1) |
| LLIYAVLPTGDVIGDSAK, | (SEQ ID NO:2) |
| LLLQQVSLPELPGEYSMK, | (SEQ ID NO:3) |
| QGLLPVLESFK, | (SEQ ID NO:4) |
| LLDNWDSVTSTFSK, | (SEQ ID NO:5) |
| KEPSQGTTTFAVTSILR, | (SEQ ID NO:6) |
| VFAIPPSFASIFLTK, | (SEQ ID NO:7) |
| QEPSQGTTTFAVTSILR, | (SEQ ID NO:8) |
| WLQGSQELPR, | (SEQ ID NO:9) |
| LWAYLTIQQLLEQTVSASDADQQALR, | (SEQ ID NO:10) |
| AEAQAQYSAAVAK, | (SEQ ID NO:11) |
| YSQPEDSLIPFFEITVPESQLTVSQFTLPK, | (SEQ ID NO:12) |
| and | |
| IAIANIIDEIIEK. | (SEQ ID NO:13) |

3. The assay according to claim 1 wherein biological fluid is selected from the group consisting of urine, saliva, blood, blood products, serum, plasma, tears, cerebrospinal fluid, and lymph.

4. The assay according to claim 3 wherein the biological fluid is urine.

5. The assay according to claim 1 wherein the biological fluid is processed prior to analysis.

6. The assay according to claim 5 wherein the biological fluid is concentrated by membrane-based electrophoresis, TCA precipitation or acetone precipitation.

7. The assay according to claim 1 wherein proteins present in the biological fluid are digested to form peptide fragments which are detected by conducting mass spectrophotometric analysis on the digested sample.

8. The assay according to claim 1 wherein the subject is a human.

9. An isolated peptide marker detectable in a biological sample of a subject and being indicative of diabetes or a predisposition to diabetes in a subject comprising one or more of the following amino acid sequences:

| | |
|---|---|
| AYIFIDEAHITQALIWLSQR, | (SEQ ID NO:1) |
| LLIYAVLPTGDVIGDSAK, | (SEQ ID NO:2) |
| LLLQQVSLPELPGEYSMK, | (SEQ ID NO:3) |
| QGLLPVLESFK, | (SEQ ID NO:4) |
| LLDNWDSVTSTFSK, | (SEQ ID NO:5) |
| KEPSQGTTTFAVTSILR, | (SEQ ID NO:6) |
| VFAIPPSFASIFLTK, | (SEQ ID NO:7) |
| QEPSQGTTTFAVTSILR, | (SEQ ID NO:8) |
| WLQGSQELPR, | (SEQ ID NO:9) |
| LWAYLTIQQLLEQTVSASDADQQALR, | (SEQ ID NO:10) |
| AEAQAQYSAAVAK, | (SEQ ID NO:11) |
| YSQPEDSLIPFFEITVPESQLTVSQFTLPK, | (SEQ ID NO:12) |
| or | |
| IAIANIIDEIIEK. | (SEQ ID NO:13) |

10. An isolated antibody directed to peptide marker according to claim 9.

11. The antibody according to claim 10 being a polyclonal antibody.

12. The antibody according to claim 10 being a monoclonal antibody.

13. The antibody according claim 10 being detectably labelled.

14. An assay for testing a subject for diabetes or a predisposition to diabetes comprising:
obtaining a urine sample from a subject;
concentrating the urine sample;
digesting proteins present in the concentrated urine sample to form peptides;
optionally, separating the peptides; and
analysing the peptides for the presence of one or marker peptides having an amino acid sequence of any one of SEQ ID NOS:1 to 13, wherein the presence of marker peptides having an amino acid sequence of any one of SEQ ID NOS:1 to 13 is indicative of diabetes or a predisposition to diabetes in the subject.

15. The assay according to claim 14 wherein the peptides are detected using an antibody directed to a marker peptide having an amino acid sequence of any one of SEQ ID NOS:1 to 13.
